(19) United States
(12) Patent Application Publication
(10) Pub. No.: US 2022/0061745 A1
(43) Pub. Date: Mar. 3, 2022

(54) SYSTEM AND PROCESS FOR IMMUNE SYSTEM EVALUATION

(71) Applicant: Jose Baldera, Miami, FL (US)

(72) Inventor: Jose Baldera, Miami, FL (US)

(21) Appl. No.: 17/011,580

(22) Filed: Sep. 3, 2020

(51) Int. Cl. A61B 5/00; A61B 5/0205; A61B 5/145; G16H 50/30

(52) U.S. Cl. CPC A61B 5/41; A61B 5/02055; A61B 5/14546; A61B 5/021; A61B 5/0022; A61B 5/6898; G16H 50/30; A61B 5/486

(57) ABSTRACT

A system and process for immune system evaluation includes sensors such as a blood analysis sensor, a computing device communicably connected with a communications network, an application configured for: detecting a current status and a current health of the patient's immune system, detecting a percentage of the patient's immune system that is currently operating, identifying vitamins and minerals that are currently in low quantities in the patient's blood, such that replacement of said vitamins and minerals would strengthen the patient's immune system, providing product purchase suggestions to the patient based on the vitamins and minerals that were identified above for strengthening the patient's immune system; and executing an online sale of the vitamins and minerals that were identified above for strengthening the patient's immune system.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The claimed subject matter relates to medical diagnostic systems and processes for immune system evaluation, and more specifically, the claimed subject matter relates to immune system evaluation using sensors and tests compatible with computing devices.

### BACKGROUND

The current state of immune system testing in the United States of America can be traced back to 1915 when infection rates of syphilis and gonorrhea were seeing unprecedented growth. Over the course of the century since then, significant growth in testing has been made, allowing for more accurate, rapid, and convenient testing. While the growth in this space can be viewed as a substantial response to a dire need, it has not yet caught up to the present-day state of medical diagnosis and testing in other areas such as food sensitivity, sexually transmitted infection testing, etc. Many of the shortfalls, specifically in the immune system testing space, are widely felt by consumer across this country and the world at large. In today's world, consumers desire, and many have come to expect, to be provided with extremely convenient, cost-effective, and valuable solutions to the medical problems they may face. Unfortunately, the present state of immune system testing does not meet these marks.

Currently, an individual looking to gather information about their immune system, and the information and materials to respond accordingly, is left with only 3 major options—(1) visit a medical professional to have blood drawn or, for women who are pregnant, prenatal testing; (2) seek out and participate in online questionnaires; or, (3) seek out and purchase an at-home immunity testing kit. While some, or perhaps all, of the options appear to be sufficient to produce the results a person may desire, considering the present state of technology both inside and outside of the medical space, they fail to really address the needs of the market.

A visit to a medical professional is a requirement at some point in everyone's life. While that may be true, these visits can be extremely costly, time consuming, and may cause potentially unnecessary anxiety or stress to the patient. In addition, these burdens are felt each time the consumer questions their immune system health. At that point they will be required to return to the medical professional's office or lab for an updated analysis. Online questionnaires may alleviate some of these burdens through convenience and reduced cost, but they often fall short in accuracy and typically fail to provide meaningful insights because of the inaccuracies. These inaccuracies are all but guaranteed due to the nature of online questionnaires. Factors such as misinformation, misunderstanding, mistakes, and blatant lying can completely render this option useless. The third and final of the major options, at-home testing, presents similar issues to the medical professional visit. For a comprehensive at-home immunity test, a consumer may end up spending upwards of $600 on a test that they must conduct themselves. Because of this, these tests are also susceptible to error in ways similar to online questionnaires. Many of these tests also include questionnaires which present the potential problems discussed above, but in addition present the risk of improper administration of the test, which may cause the lab to return inconclusive, or at times incorrect, results. In these cases, the consumer is out of pocket the money for the first test and has lost the time spent waiting on results. To reach their desired results, they will have to either purchase another test and wait the required time or consider taking a different approach. While a medical professional may be contracted to come to the consumer and conduct the test, this significantly increases the cost and reduces the convenience of at-home tests by requiring them to be done on the medical professional's time. In addition, like the results of an in-person exam administered at a medical professional's office or lab, these tests are single-use and fail to provide a method of tracking or monitoring immune system health over time. Many companies providing these test kits offer subscription-based services to address the immediately aforementioned problem, but this approach significantly increases the amount of time and money one must invest. In the event that the user of these test kits carries out each step correctly, they are still often left blind regarding how to respond to their results and where to get proper treatment, an outcome similar to the that experienced after using an online questionnaire.

As a result of at least the aforementioned shortfalls in the prior art, the need exists for a convenient, affordable, and accurate system and process for immune system evaluation. Specifically, the need exists for an immune system evaluation and process that facilitates the maintenance and monitoring of a person's immune system using sensors and monitors that are compatible with computing devices and their corresponding software.

### BRIEF SUMMARY

In one embodiment, a system and process for immune system evaluation is disclosed. The system comprises one or more sensors including a blood analysis sensor, a computing device communicably connected with a communications network, wherein the one or more sensors are communicably connected to the mobile computing device, an application executing on the computing device, the application configured for: detecting, via the one or more sensors, a current status and a current health of the patient's immune system, detecting, via the one or more sensors, a percentage of the patient's immune system that is currently operating, identifying, via the one or more sensors, vitamins and minerals that are currently in low quantities in the patient's blood, such that replacement of said vitamins and minerals would strengthen the patient's immune system, providing product purchase suggestions to the patient based on the vitamins and minerals that were identified above for strengthening the patient's immune system; and executing an online sale of the vitamins and minerals that were identified above for strengthening the patient's immune system.

Additional aspects of the claimed subject matter will be set forth in part in the description which follows, and in part will be obvious from the description, or may be learned by practice of the claimed subject matter. The aspects of the claimed subject matter will be realized and attained by means of the elements and combinations particularly pointed out in the appended claims. It is to be understood that both the foregoing general description and the following detailed description are exemplary and explanatory only and are not restrictive of the disclosed subject matter, as claimed.

### BRIEF DESCIPTION OF THE DRAWINGS

FIG. 1 is a block diagram illustrating the network architecture of a system for immune system evaluation, in accordance with one embodiment.

FIG. 2 is a block diagram showing the data flow of a system and process for immune system evaluation, according to one embodiment.

FIG. 3 is a flow chart depicting the general control flow of a process for immune system evaluation, according to one embodiment.

FIG. 4 is a block diagram depicting a system including an example computing system and other computing devices.

### DETAILED DESCRIPTION

The disclosed embodiments improve upon the issues identified within the prior art by facilitating and streamlining the process for immune system evaluation. Specifically, the disclosed embodiments improve over the prior art by providing a turnkey solution for the potentially immunocompromised or those with weakened immune systems to gather information regarding the health of their immune system and respond to such information accordingly. The disclosed embodiments specifically improve the “information gathering” portion of the immune system evaluation process by providing users with a thorough, convenient, time and cost-efficient method of immune system evaluation. The claimed embodiments provide a more convenient method by allowing users to complete the entire process in the comfort of a place of their choosing, from their mobile device, using sensors. The sensors may include but are not limited to heart rate monitors, blood pressure sensors, blood sensors, and the like. The disclosed embodiments further improve convenience by providing an environment that allows multiple uses of the same materials, sensors, and devices, thereby reducing the time and money investments, as well as stress factors, associated with the more conventional method of in-office testing.

The disclosed embodiments improve over the prior art regarding test result response by providing a centralized location where test results can be viewed and considered in conjunction with offerings of potentially necessary remedies or solutions. In addition to linking the results to the appropriate next steps, an offering virtually absent in the present state of immune system evaluation, the present embodiments further allow the user to act on such recommendations, facilitating the process of selecting and purchasing the appropriate vitamins, minerals, or other products to respond to test results.

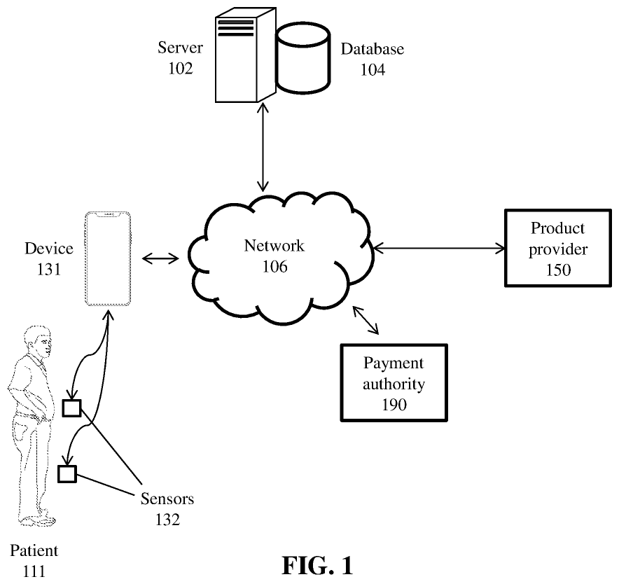

Referring now to the drawing figures in which like reference designators refer to like elements, there is shown in FIG. 1 an illustration of a block diagram showing the network architecture of a system 100 and a method for immune system evaluation. A prominent element of FIG. 1 is the server, 102, associated with repository or database 104 and further communicatively coupled with network 106, which can be a circuit switched network, such as the Public Service Telephone Network (PTSN), or a packet switched network, such as the Internet or the World Wide Web, the global telephone network, a cellular network, a mobile communications network, or any combination of the above. Server 102 is a central controller or operator for functionality of the disclosed embodiments.

FIG. 1 includes computing device 131, which may be a mobile computing device such as a smart phone, mobile phone, tablet computer, handheld computer, laptop, or the like. In another embodiment, computing device 131 may be a workstation, desktop computer, server, all-in-one computer, or the like. In yet another embodiment, computing device 131 may be an AR or VR system that may include display screens, headsets, heads-up displays, helmet mounted display screens, tracking devices, tracking lighthouses, or the like. Computing device 131 corresponds to a user or patient 111 who may be seeking to obtain information regarding immune system health and the appropriate response to said information. Computing device 131 may be communicatively coupled with network 106 in a wired or wireless fashion.

FIG. 1 further includes sensors 132 which may be communicatively coupled with device 131 either in a wired fashion or wirelessly. Sensors 132 may be any of the variety of sensors commonly used to test the immune system, including but not limited to a blood analysis sensor, a pulse oximetry sensor, a blood pressure sensor, a heart rate sensor, a temperature sensor, a glucose monitor and a movement monitor. A blood analysis sensor may be based on swept-wavelength infrared laser absorption spectroscopy that is well known in the art. Each molecule has a unique absorption spectrum that can be used for species and concentration identification. A swept-wavelength laser shines into the patient's tissue with capillaries and interacts with target molecules that have resonant absorption. The reflected light carrying molecule species and concentration information is collected and a signal analysis is carried out.

FIG. 1 also depicts a product provider 150 and payment authority 190 which may communicate with the user via network 106. Product provider 150 may be one or a variety of any business engaged in the sale or provision of vitamins, minerals, medicines, or any other product that may be relevant in the improvement or maintenance of the human immune system. Payment authority 190 acts to effectuate payments by the user for the goods and/or services provided by product provider 150. During a sales transaction, server 102 may interface with payment authority 190 to effectuate payment. In one embodiment, payment authority 190 is a payment gateway, which is an e-commerce Application Service Provider (ASP) service that authorizes and processes payments from one party to another. The payment authority 190 may accept payment via the use of purchase cards, i.e., credit cards, charge cards, bank cards, gift cards, account cards, etc.

Associated with the server 102 in FIG. 1 is a database or repository 104 which maybe a relational database comprising a Structured Query Language (SQL) database stored in a SQL server. Device 131 may also include its own database. The repository 104 serves data from a database, which is a repository for data used by server 102 and device 131 during operation of the disclosed embodiments. Database 104 may be distributed over one or more nodes or locations that are connected via network 106.

The database 104 may include a user record for each user or patient 111. A user record may include contact/identifying information for the user (name, address, telephone number(s), email address, etc.), contact/identifying information for an emergency contact of the user, electronic payment information for the user, information pertaining to the purchases made by the user, sales transaction data associated with the user, user healthcare data, etc. A user record may also include demographic data for each user, such as age, sex, income data, race, color, marital status, etc. A user record may also include a unique identifier for one or more medical professionals associated with the user.

A user record may also record any data collected by the sensors 132 used to test the immune system, including but not limited to a blood analysis sensor, a pulse oximetry sensor, a blood pressure sensor, a heart rate sensor, a temperature sensor, a glucose monitor and a movement monitor. Said data collected by the sensors 132 may include blood analysis data (including blood panel results or blood panel data), pulse oximetry data, blood pressure data, heart rate data (including resting heart rate, walking heart rate, heart variability, EKG data, etc.), temperature data, blood glucose data and a movement data. Said data collected by the sensors 132 may also include measuring levels of infection-fighting proteins, blood cells and immune system cells in the patient's blood. This includes measuring interferons in the blood, cytokines in the blood, major histocompatibility complex (MHC) antigens in the blood, and the like.

Said data collected by the sensors 132 may also include measuring levels of blood cells (such as hematocrit level) and immune system cells in the patient's blood, such as T-cells, lymphocytes, immunoglobin (IgA, IgC, IgM, IgE, IgD), and the like. Said data collected by the sensors 132 may also include measuring levels of vitamins and minerals in the blood, including vitamin D, iron, serum ferritin, hemoglobin, transferrin saturation, total iron-binding capacity, unsaturated iron-binding capacity, active B12, total B12, folate, calcium, vitamin K1, alkaline phosphatase, calcium with creatinine, Vitamins A, C, E and beta carotene, glucose, BUN, creatinine, serum, bun/creatinine ratio, glomerular filtration (eGFR), total globulin, albumin, total bilirubin, albumin/globulin ratio, total cholesterol/HDL ratio, and the like.

Sales transaction data may include one or more product/service identifiers (such as SKUs), one or more product/service amounts, buyer contact/identifying information, brick and mortar outlet information, and electronic payment information. In one embodiment, electronic payment information may comprise buyer contact/identifying information and any data garnered from a purchase card (i.e., purchase card data), as well as any authentication information that accompanies the purchase card. Purchase card data may comprise any data garnered from a purchase card and any authentication information that accompanies the purchase card. In one embodiment, electronic payment information may comprise user login data, such as a login name and password, or authentication information, which is used to access an account that is used to make a payment.

The database 104 may also include a vendor record for each product provider 150. A vendor record may include contact/identifying information for the vendor (name, address, telephone number(s), email address, etc.), information pertaining to products offered by the vendor, electronic payment information for the vendor (for receiving payment for goods and services), sales transaction data associated with the vendor, etc. A vendor record may also include data defined by the product provider, such as such as product pricing, offers/deals, product availability, vendor ratings and reviews, special delivery options, associated vendors, or any other information deemed relevant by the vendor.

FIG. 1 shows an embodiment wherein networked computing device 131 interacts with server 102 and repository 104 over the network 106. It should be noted that although FIG. 1 shows only the networked computers 131 and 102, the system of the disclosed embodiments supports any number of networked computing devices connected via network 106. Further, server 102, and unit 131 include program logic such as computer programs, mobile applications, executable files or computer instructions (including computer source code, scripting language code or interpreted language code that may be compiled to produce an executable file or that may be interpreted at run-time) that perform various functions of the disclosed embodiments.

Note that although server 102 is shown as a single and independent entity, in one embodiment, the functions of server 102 may be integrated with another entity, such as device 131. Further, server 102 and its functionality, according to a preferred embodiment, can be realized in a centralized fashion in one computer system or in a distributed fashion wherein different elements are spread across several interconnected computer systems.

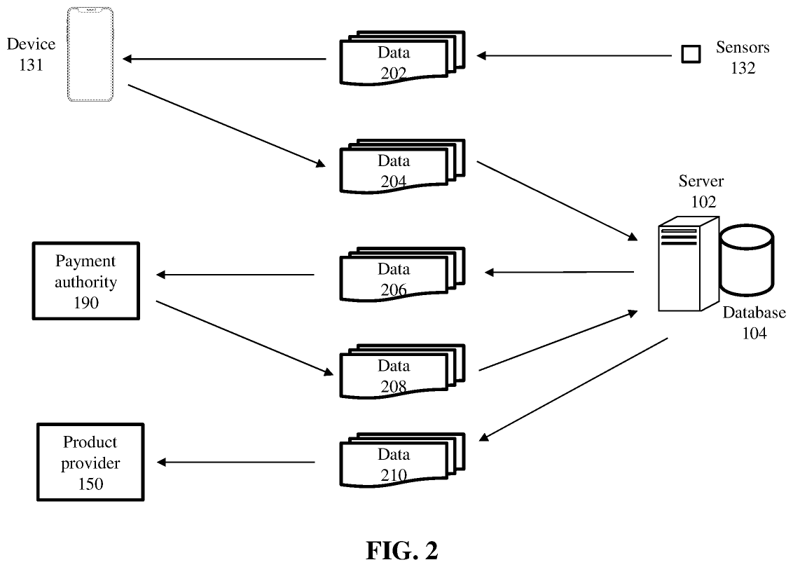

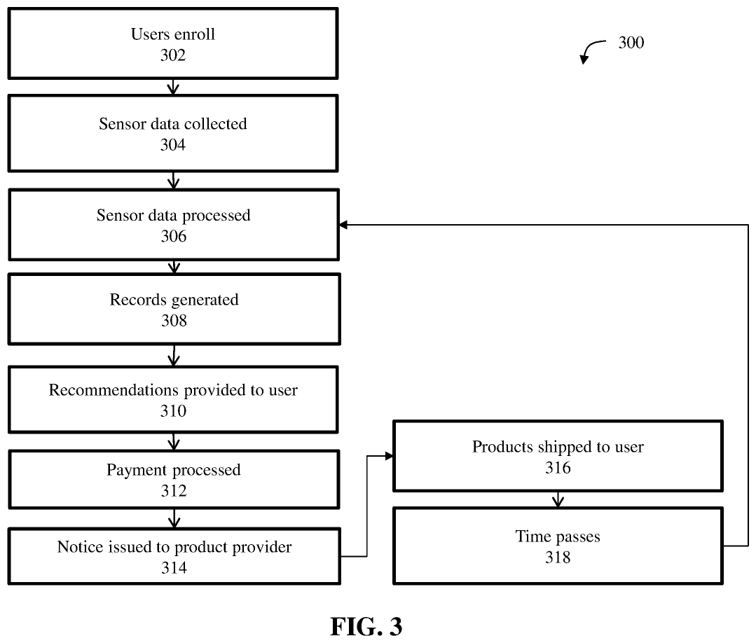

The process for immune system evaluation will now be described with reference to FIGS. 2 and 3 below. FIG. 2 depicts the data flow of the process for immune system evaluation while FIG. 3 depicts the control flow of the process for immune system evaluation, according to one embodiment.

The process of the disclosed embodiments begins with step 302 (see FIG. 3) where patient 111 enrolls, sending the data relevant to enrollment to server 102. In the course of enrolling or registering, the patient may enter data into their device by manually entering data into an application via keypad, touchpad, or via voice. In the course of enrolling or registering, the patient may enter any data that may be stored in a user record, as defined above. Also, while enrolling or registering, the server 102 may generate a user record for each registering user and store the user record in an attached database, such as database 104.

Once enrolled, patient 111 device 131 collects data 202 (from the sensors 132) in step 304. The data collected from the sensors includes blood analysis data (including blood panel results or blood panel data), pulse oximetry data, blood pressure data, heart rate data (including resting heart rate, walking heart rate, heart variability, EKG data, etc.), temperature data, blood glucose data and a movement data. Said data is stored in a user record associated with the patient 111. Said data collected by the sensors 132 may also include measuring levels of infection-fighting proteins, blood cells and immune system cells in the patient's blood. This includes measuring interferons in the blood, cytokines in the blood, major histocompatibility complex (MHC) antigens in the blood, and the like.

Said data collected by the sensors 132 may also include measuring levels of blood cells (such as hematocrit level) and immune system cells in the patient's blood, such as T-cells, lymphocytes, immunoglobin (IgA, IgC, IgM, IgE, IgD), and the like. Said data collected by the sensors 132 may also include measuring levels of vitamins and minerals in the blood, including vitamin D, iron, serum ferritin, hemoglobin, transferrin saturation, total iron-binding capacity, unsaturated iron-binding capacity, active B12, total B12, folate, calcium, vitamin K1, alkaline phosphatase, calcium with creatinine, Vitamins A, C, E and beta carotene, glucose, BUN, creatinine, serum, bun/creatinine ratio, glomerular filtration (eGFR), total globulin, albumin, total bilirubin, albumin/globulin ratio, total cholesterol/HDL ratio, and the like.

In step 306, the data collected in step 304 is processed. The step 306 of processing the data may comprise multiple steps including detecting, via the one or more sensors, a current status and a current health of the patient's immune system comprises measuring levels of infection-fighting proteins, blood cells and immune system cells in the patient's blood. The step 306 of processing the data may further comprise detecting, via the one or more sensors, a percentage of the patient's immune system that is currently operating comprises comparing the levels of infection-fighting proteins, blood cells and immune system cells in the patient's blood that were measured to normal levels (stored in database 104 as a reference) of infection-fighting proteins, blood cells and immune system cells.

The step 306 of processing the data may further comprise identifying, via the one or more sensors, vitamins and minerals that are currently in low quantities in the patient's blood comprises measuring levels of a predefined set of vitamins and minerals in the patient's blood and comparing said levels that were measured to normal levels (stored in database 104 as a reference) of the predefined set of vitamins and minerals.

The step 306 of processing the data may further comprise accessing previously stored data collected from the sensors, creating a Markov model of the vitamins and minerals quantities in the patient's blood using said sensor readings, and identifying, via the Markov model, which vitamins and minerals will be (in the future or near future) low or at unhealthy levels, as per the future prediction of the Markov model of the levels of vitamins and minerals, as compared to normal levels (stored in database 104 as a reference) of the predefined set of vitamins and minerals.

The step 306 of processing the data may further comprise accessing previously stored data collected from the sensors, creating a Markov model of the patient's immune system using said sensor readings, and determining, via the Markov model, if the status and health of the patient's immune system will be (in the future or near future) low or unhealthy, as per the future prediction of the Markov model of the levels of infection-fighting proteins, blood cells and immune system cells in the patient's blood (wherein said levels are compared normal levels stored in database 104 as a reference). Said Markov model may also identify those vitamins and minerals that affect said status and health of the patient's immune system.

The step 306 of processing the data may further comprise accessing previously stored data collected from the sensors, creating a Markov model of the patient's immune system using said sensor readings, and determining, via the Markov model, if the percentage of the patient's immune system that will be (in the future or near future) operating will be low or at unhealthy levels, as per the future prediction of the Markov model of infection-fighting proteins, blood cells and immune system cells in the patient's blood (wherein said levels are compared normal levels stored in database 104 as a reference). Said Markov model may also identify those vitamins and minerals that affect said percentage of the patient's immune system.

In step 308, the data that was collected and generated in the previous steps is sent to server 102 as a data packet 204 and a record is generated in database 104 including said data from the data packet 204 in association with the user record of the patient 111. In step 310, the server analyzes the data that was received and stored and provides product recommendations or product purchase suggestions to the patient 111 based on the vitamins and minerals that were identified for strengthening the patient's immune system. Providing product purchase suggestions further comprises providing information regarding potential impacts and recommended dosage of said product purchase suggestions. The disclosed embodiments are configured to allow the user to select or selections based on the recommendations provided in step 310.

Once a selection is made the payment is processed in step 312. In step 312, electronic payment information presented by the patient 111 when enrolling (or at a later time) is transmitted by server 102 to payment authority 190 (as data 206) for processing. The payment authority 190 processes the electronic payment information and verifies whether payment has been effectuated. If so, in step 314, the payment authority 190 may send a verification message (data 208) to the server 102 thereby verifying that the payment has been effectuated. Once the product provider has been notified, the product provider ships the product(s) to the patient 111 in step 316 using the information provided in step 302.

Given the multi-use nature of the disclosed embodiments, the user may find that as time passes, as shown in step 318, their desire or need for more accurate and up to date information may increase. Alternatively, their needs may entirely change because of previous treatment or lifestyle changes. As FIG. 3 shows, time passes in step 318 and the user may return to complete the process again, this time initiating the process at step 304 given that the user will have previously enrolled in step 302.

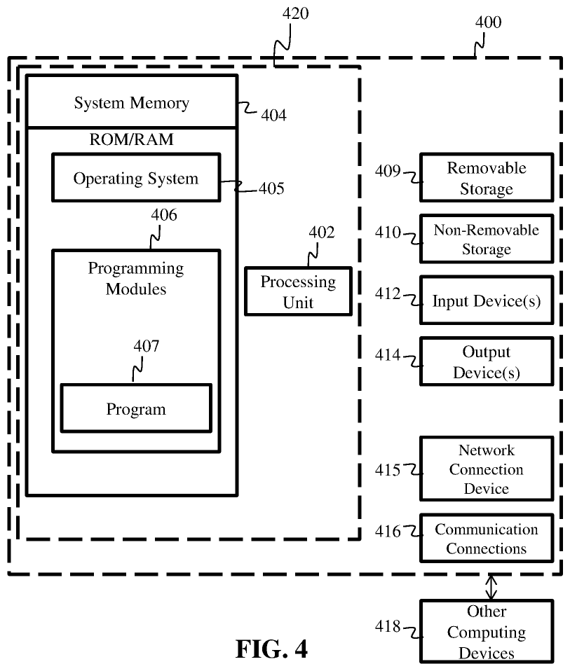

FIG. 4 is a block diagram of a system including an example computing device 400 and other computing devices. Consistent with the embodiments described herein, the actions performed by 131, 102 may be implemented in a computing device, such as the computing device 400 of FIG. 4. Any suitable combination of hardware, software, or firmware may be used to implement the computing device 400. The system, device, and processors are examples and other systems, devices, and processors may comprise the computing device. Furthermore, computing device 400 may comprise an operating environment for system 100 and process 300, as described above. Process 300 may operate in other environments and are not limited to computing device 400.

With reference to FIG. 4, a system consistent with an embodiment may include a plurality of computing devices, such as computing device 400. In a basic configuration, computing device 400 may include at least one processing unit 402 and a system memory 404. Depending on the configuration and type of computing device, system memory 404 may comprise, but is not limited to, volatile (e.g. random-access memory (RAM)), non-volatile (e.g. read-only memory (ROM)), flash memory, or any combination or memory. System memory 404 may include operating system 405, and one or more programming modules 406. Operating system 405, for example, may be suitable for controlling computing device 400′s operation. In one embodiment, programming modules 406 may include, for example, a program module 407 for executing the actions of 131, 102. Furthermore, embodiments may be practiced in conjunction with a graphics library, other operating systems, or any other application program and is not limited to any application or system. This basic configuration is illustrated in FIG. 4 by those components within a dashed line 420.

Computing device 400 may have additional features or functionality. For example, computing device 400 may also include additional data storage devices (removable and/or non-removable) such as, for example, magnetic disks, optical disks, or tape. Such additional storage is illustrated in FIG. 4 by a removable storage 409 and a non-removable storage 410. Computer storage media may include volatile and nonvolatile, removable, and non-removable media implemented in any method or technology for storage of information, such as computer readable instructions, data structures, program modules, or other data. System memory 404, removable storage 409, and non-removable storage 410 are all computer storage media examples (i.e. memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 400. Any such computer storage media may be part of device 400. Computing device 400 may also have input device(s) 412 such as a keyboard, a mouse, a pen, a sound input device, a camera, a touch input device, etc. Output device(s) 414 such as a display, speakers, a printer, etc. may also be included. Computing device 400 may also include a vibration device capable of initiating a vibration in the device on command, such as a mechanical vibrator or a vibrating alert motor. The devices are only examples, and other devices may be added or substituted.

Computing device 400 may also contain a network connection device 415 that may allow device 400 to communicate with other computing devices 418, such as over a network in a distributed computing environment, for example, an intranet or the Internet. Device 415 may be a wired or wireless network interface controller, a network interface card, a network interface device, a network adapter, or a LAN adapter. Device 415 allows for a communication connection 416 for communicating with other computing devices 418. Communication connection 416 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, several program modules and data files may be stored in system memory 404, including operating system 405. While executing on processing unit 402, programming modules 406 (e.g. program module 407) may perform processes including, for example, one or more of the stages of the process 300 as described above. The processes are examples, and processing unit 402 may perform other processes. Other programming modules that may be used in accordance with embodiments herein may include electronic mail and contacts applications, word processing applications, spreadsheet applications, database applications, slide presentation applications, drawing or computer-aided application programs, etc.

Generally, consistent with embodiments herein, program modules may include routines, programs, components, data structures, and other types of structures that may perform particular tasks or that may implement particular abstract data types. Moreover, embodiments herein may be practiced with other computer system configurations, including hand-held devices, multiprocessor systems, microprocessor-based or programmable consumer electronics, minicomputers, mainframe computers, and the like. Embodiments herein may also be practiced in distributed computing environments where tasks are performed by remote processing devices that are linked through a communications network. In a distributed computing environment, program modules may be in both local and remote memory storage devices.

Furthermore, embodiments herein may be practiced in an electrical circuit comprising discrete electronic elements, packaged or integrated electronic chips containing logic gates, a circuit utilizing a microprocessor, or on a single chip (such as a System on Chip) containing electronic elements or microprocessors. Embodiments herein may also be practiced using other technologies capable of performing logical operations such as, for example, AND, OR, and NOT, including but not limited to mechanical, optical, fluidic, and quantum technologies. In addition, embodiments herein may be practiced within a general-purpose computer or in any other circuits or systems.

Embodiments herein, for example, are described above with reference to block diagrams and/or operational illustrations of methods, systems, and computer program products according to said embodiments. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments have been described, other embodiments may exist. Furthermore, although embodiments herein have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages, and/or inserting or deleting stages, without departing from the claimed subject matter.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A system for evaluating a patient's immune system, the system comprising:
1) one or more sensors, including a blood analysis sensor;
2) a computing device communicably connected with a communications network, wherein the one or more sensors are communicably connected to the mobile computing device;
3) an application executing on the computing device, the application configured for:
a) detecting, via the one or more sensors, a current status and a current health of the patient's immune system;
b) detecting, via the one or more sensors, a percentage of the patient's immune system that is currently operating;
c) identifying, via the one or more sensors, vitamins and minerals that are currently in low quantities in the patient's blood, such that replacement of said vitamins and minerals would strengthen the patient's immune system;
d) providing product purchase suggestions to the patient based on the vitamins and minerals that were identified for strengthening the patient's immune system; and
e) executing an online sale of the vitamins and minerals that were identified for strengthening the patient's immune system.

2. The system of claim 1, repeating steps a) through e) at a later time.

3. The system of claim 1, wherein the one or more sensors further include one or more of a pulse oximetry sensor, a blood pressure sensor, a heart rate sensor, a temperature sensor, a glucose monitor, and a movement monitor.

4. The system of claim 3, wherein the computing device comprises a mobile computing device.

5. The system of claim 4, wherein the step of detecting, via the one or more sensors, a current status and a current health of the patient's immune system comprises measuring levels of infection-fighting proteins, blood cells and immune system cells in the patient's blood.

6. The system of claim 5, wherein the step of detecting, via the one or more sensors, a percentage of the patient's immune system that is currently operating comprises comparing the levels of infection-fighting proteins, blood cells and immune system cells in the patient's blood that were measured to normal levels of infection-fighting proteins, blood cells and immune system cells.

7. The system of claim 6, wherein the step of identifying, via the one or more sensors, vitamins and minerals that are currently in low quantities in the patient's blood comprises measuring levels of a predefined set of vitamins and minerals in the patient's blood and comparing said levels that were measured to normal levels of the predefined set of vitamins and minerals.

8. The system of claim 7, wherein the step of providing product purchase suggestions further comprises providing information regarding potential impacts and recommended dosage of said product purchase suggestions.

9. A system for evaluating a patient's immune system, the system comprising:
1) one or more sensors, including a blood analysis sensor;
2) a mobile computing device communicably connected with a communications network, wherein the one or more sensors are communicably connected to the mobile computing device;
3) a mobile application executing on the computing device, the mobile application configured for:
a) detecting, via the one or more sensors, a current status and a current health of the patient's immune system;
b) detecting, via the one or more sensors, a percentage of the patient's immune system that is currently operating;
c) identifying, via the one or more sensors, vitamins and minerals that are currently in low quantities in the patient's blood, such that replacement of said vitamins and minerals would strengthen the patient's immune system;
d) providing product purchase suggestions to the patient based on the vitamins and minerals that were identified for strengthening the patient's immune system; and
e) executing an online sale of the vitamins and minerals that were identified for strengthening the patient's immune system.

10. The system of claim 9, repeating steps a) through e) at a later time.

11. The system of claim 9, wherein the one or more sensors further include one or more of a pulse oximetry sensor, a blood pressure sensor, a heart rate sensor, a temperature sensor, a glucose monitor, and a movement monitor.

12. The system of claim 11, wherein the mobile computing device comprises a smartphone.

13. The system of claim 12, wherein the step of detecting, via the one or more sensors, a current status and a current health of the patient's immune system comprises measuring levels of infection-fighting proteins, blood cells and immune system cells in the patient's blood.

14. The system of claim 13, wherein the step of detecting, via the one or more sensors, a percentage of the patient's immune system that is currently operating comprises comparing the levels of infection-fighting proteins, blood cells and immune system cells in the patient's blood that were measured to normal levels of infection-fighting proteins, blood cells and immune system cells.

15. The system of claim 14, wherein the step of identifying, via the one or more sensors, vitamins and minerals that are currently in low quantities in the patient's blood comprises measuring levels of a predefined set of vitamins and minerals in the patient's blood and comparing said levels that were measured to normal levels of the predefined set of vitamins and minerals.

16. The system of claim 15, wherein the step of providing product purchase suggestions further comprises providing information regarding potential impacts and recommended dosage of said product purchase suggestions.

17. A system for evaluating a patient's immune system, the system comprising:
1) one or more sensors, including a blood analysis sensor;
2) a mobile computing device communicably connected with a communications network, wherein the one or more sensors are communicably connected to the mobile computing device;
3) a mobile application executing on the computing device, the mobile application configured for:
a) detecting, via the one or more sensors, levels of infection-fighting proteins, blood cells and immune system cells in the patient's blood;
b) calculating, based on the levels of infection-fighting proteins, blood cells and immune system cells in the patient's blood, a current status and a current health of the patient's immune system;
c) calculating, based on the levels of infection-fighting proteins, blood cells and immune system cells in the patient's blood, a percentage of the patient's immune system that is currently operating;
d) identifying, based on the levels of infection-fighting proteins, blood cells and immune system cells in the patient's blood, vitamins and minerals that would strengthen the patient's immune system;
e) generating a Markov model of the current status and the current health of the patient's immune system and the percentage of the patient's immune system that is currently operating, based on the levels of infection-fighting proteins, blood cells and immune system cells in the patient's blood;
f) identifying, via the Markov model, vitamins and minerals that will be in low quantities in the patient's blood, such that replacement of said vitamins and minerals would strengthen the patient's immune system;
g) providing product purchase suggestions to the patient based on the vitamins and minerals that were identified for strengthening the patient's immune system;
h) receiving confirmation from the client to purchase the vitamins and minerals that were identified for strengthening the patient's immune system; and
i) executing an online sale of the vitamins and minerals that were identified for strengthening the patient's immune system.

18. The system of claim 17, repeating steps a) through i) at a later time.

19. The system of claim 17, wherein the one or more sensors further include one or more of a pulse oximetry sensor, a blood pressure sensor, a heart rate sensor, a temperature sensor, a glucose monitor, and a movement monitor.

20. The system of claim 17, wherein the mobile computing device comprises a smartphone.
